(19) United States
(12) Patent Application Publication
(10) Pub. No.: US 2015/0248843 A1
(43) Pub. Date: Sep. 3, 2015

(54) TRAINING METHODS FOR IMPROVED ASSAYING OF PAIN IN CLINICAL TRIAL SUBJECTS

(71) Applicant: ANALGESIC SOLUTIONS, Natick, MA (US)

(72) Inventors: Nathaniel Katz, Natick, MA (US); Jeremiah J. Trudeau, Natick, MA (US)

(73) Assignee: ANALGESIC SOLUTIONS, Natick, MA (US)

(21) Appl. No.: 14/429,623

(22) PCT Filed: Oct. 11, 2013
(86) PCT No.: PCT/US2013/064542

§ 371 Date: Mar. 19, 2015

Related U.S. Application Data
(60) Provisional application No. 61/713,032, filed on Oct. 12, 2012.

(51) Int. Cl. G09B 19/00; A61B 5/00

(52) U.S. Cl. CPC G09B 19/00; A61B 5/4824; A61B 5/0053

(57) ABSTRACT

Provided are methods for training subjects to report pain, and for identifying accurate pain reporting subjects prior to or subsequent to training. The methods of the invention generally involve: determining the reported pain threshold and tolerance levels of the subject in response to an evoked pain stimulus; determining the reported pain of the subject in response to a natural index pain using a standard pain reporting scale; determining the response profile of the subject to noxious stimuli using a standard pain reporting scale, wherein the noxious stimuli intensity are between the pain threshold and tolerance levels of the subject; and determining the pain reporting accuracy and/or reliability of the subject.

### BACKGROUND OF THE INVENTION

Subject self-reporting (verbal or written) of pain levels is the source of virtually all important efficacy outcome data in clinical trials for analgesics. With the exception of physically observable changes such as blood pressure or pupil dilation, which are unsuitable primary measures of pain, researchers generally rely upon a subject's subjective self-reporting of their pain experience (Patient Reported Outcome, PRO). Thus, subject self-reporting of pain is an important contributor to treatment group differences and variation, both of which affect clinical trial sensitivity. Indeed, double-blind clinical trials for analgesics have often failed due to distorted or ‘noisy’ pain reports from subjects.

Much effort has gone into maximizing the assay sensitivity of clinical trials for potential analgesics. Increasing assay sensitivity has the obvious benefit of reducing sample size requirements for clinical trials, thus allowing the same information to be derived by experimentation on fewer human subjects. This, in turn, reduces cost and time to conduct the trial, and decreases the likelihood of false negative trials (i.e., when an efficacious analgesic fails to separate from placebo). To accurately discriminate between an effective analgesic compound and placebo a clinical study requires adequate sensitivity and statistical power.

Calculations of statistical power involve two essential components: treatment group differences (difference in mean pain scores between each group) and variation of those pain scores. Many factors can contribute to each of these, such as a subject's pre-treatment characteristics, treatment dosage, study design factors, precision of outcome measures, and, of course, actual treatment efficacy. Researchers have explored practices and procedures to maximize treatment group differences and minimize variations, mainly by focusing their efforts on optimizing study designs and outcome measures. However, none of these optimizations have focused on the source of the data: the subjects themselves.

Pain is a subjective experience that is a function of both physical sensations and psychological processes. Therefore, for the same level of pain-producing physical stimuli (e.g., experimental pain, arthritic joint, bone metastasis, etc.), there may be important individual differences in the pain experience. When subjected to the same pain-producing stimulus, some subjects may report their pain levels reliably and precisely, while others may vary wildly in their reports of pain for the same experience. Importantly, individuals with large pain variation are more likely to respond to placebo or respond well to both the analgesic and the placebo. Such individuals not only introduce “noise” by the large degree of variation in their pain scores, but also decrease the ability of the trial to discriminate between treatment groups due to their greater tendency to experience spontaneous resolution or placebo responses in a clinical trial. Subjects with inconsistent pain reports also tend to continue to be inconsistent over time.

Much of the research concerned with subject pain reporting seeks to validate particular assessment scales, or the utility of one method of measurement relative to another. Other approaches are focused on statistical or methodological manipulation of pain reports, such as training people to make their reports relative to given anchor points (a method called “Constrained Scaling”) or constructing scales that adapt to individual reporters' biases and nuances (an approach termed “Master Scaling”). However, these procedures are too cumbersome or impractical for implementation in clinical trials. Moreover, it is unlikely that one single scale takes into account all factors associated with pain reporting reliability or lack thereof.

Accordingly, there is a need in the art for improved methods of assaying pain reporting subjects, especially methods that can identify accurate pain reporting subjects.

### SUMMARY OF THE INVENTION

The present invention provides methods for training subjects to report pain, and for identifying accurate pain reporting subjects prior to or subsequent to training. The methods of the invention generally involve: determining the reported pain threshold and tolerance levels of the subject in response to an evoked pain stimulus; determining the reported pain of the subject in response to a natural index pain using a standard pain reporting scale; determining the response profile of the subject to noxious stimuli using a standard pain reporting scale, wherein the noxious stimuli intensity are between the pain threshold and tolerance levels of the subject; and determining the pain reporting accuracy and/or reliability of the subject.

The methods of the invention improve the accuracy of pain reporting of subjects and also allow for identification of those subjects that are accurate pain reporters. Such methods are particularly useful for clinical trials of analgesics where the training and selection of accurate pain reporting subjects improves the statistical power and accuracy of the clinical trial results.

Accordingly in one aspect the invention provides a method of training a subject to report pain comprising: a) determining the reported pain threshold and tolerance levels of the subject in response to an evoked pain stimulus; b) determining the reported pain of the subject in response to a natural index pain using a standard pain reporting scale; c) determining the response profile of the subject to noxious stimuli using a standard pain reporting scale, wherein the noxious stimuli intensity are between the pain threshold and tolerance levels of the subject; d) determining the pain reporting accuracy and/or reliability of the subject by analysis of the data collected in (a), (b), and (c); e) providing instructional feedback to the subject regarding the accuracy and reliability of their pain reporting; and f) repeating steps (a) to (e) one or more times. In certain embodiments of the method, step (f) is repeated until a desired reporting accuracy is achieved. In other embodiments, step (f) is repeated a predetermined number of times.

In another aspect the invention provides a method of identifying an accurate pain reporting subject: a) determining the reported pain threshold and tolerance levels of the subject in response to an evoked pain stimulus; b) determining the reported pain of the subject in response to a natural index pain using a standard pain reporting scale; c) determining the response profile of the subject noxious stimuli using a standard pain reporting scale, wherein the noxious stimuli evoke pain that is between the pain threshold and tolerance levels of the subject; d) determining the pain reporting accuracy and/or reliability of the subject by analysis of data obtained in (a), (b), and (c), wherein an accurate pain reporting subject is identified by having a pain reporting accuracy and/or reliability above a desired threshold.

In certain embodiments of the methods disclosed herein, the pain threshold and tolerance levels of the subject are determined in response to a mechanical pressure or thermal stimulus.

In certain embodiments of the methods disclosed herein, the index pain is knee pain from osteoarthritis.

In certain embodiments of the methods disclosed herein, the noxious stimuli include mechanical pressure or thermal stimuli.

In certain embodiments of the methods disclosed herein, the noxious stimuli are applied in a random order of intensity.

In certain embodiments of the methods disclosed herein, the noxious stimuli are applied in discreet interval levels, evenly spaced between the subject's threshold and tolerance levels. In one particular embodiment, the noxious stimuli are applied in 5 to 9 interval levels. In another particular embodiment, each interval level of noxious stimuli is applied between 3 and 7 times to the subject during a single session.

In certain embodiments of the methods disclosed herein, the standard pain reporting scale is a numerical rating scale (NRS) or visual analog scale (VAS).

In certain embodiments of the methods disclosed herein, the pain reporting accuracy and/or reliability of the subject is determined using a the Coefficient of Variation, Intraclass Correlation Coefficient, R² curve fit statistic from a least squares fit to psychophysical function, and/or the Residual between the predicted and actual pain ratings using a ‘triangulation’ method.

In certain embodiments of the methods disclosed herein, an accurate pain reporting subject is identified by having a Coefficient of Variation of less than 1, an Intraclass Correlation Coefficient of greater than 0.8, an R² of greater than 0.5, and/or a triangulation residual of less than 20% of the range of the response scale being used.

### BRIEF DESCRIPTION OF THE DRAWINGS

FIG. 1 depicts an example of a psychophysical subject profile, plotting the reported pain intensity against the applied pressure stimulus.

FIG. 2 depicts a plot of the consistency of pain reporting of a subject quantified by the residual between the point where index pain standard scale and Pain Match ratings intersect, and a vertical line dropped to the psychophysical function.

### DETAILED DESCRIPTION

### I. Definitions

As used herein, the term “natural index pain” or “index pain” refers to the natural pain perceived by a subject as a result of a disease/disorder, injury and/or surgical procedure. Exemplary index pain includes, without limitation, knee pain from osteoarthritis.

### II. Pain Training Overview

In one aspect, the present invention provides methods of training a subject to report pain. Such methods generally involve: determining the reported pain threshold and tolerance levels of the subject in response to an evoked pain stimulus; determining the reported pain of the subject in response to a natural index pain using a standard pain reporting scale; determining the response profile of the subject to noxious stimuli using a standard pain reporting scale, wherein the noxious stimuli intensity are between the pain threshold and tolerance levels of the subject; determining the pain reporting accuracy and/or reliability of the subject; and providing instructional feedback to the subject regarding the accuracy and reliability of their pain reporting.

In certain embodiments, the methods of the invention involve Evoked Pain Training. Evoked Pain Training is a technique by which potential subjects for a clinical trial are trained on the use of pain reporting scales and attention to their personal pain states by repeated exposures to evoked pain stimuli and report of their pain experiences. Subjects are provided with feedback on their performance and undergo multiple cycles of training and performance that is quantified on multiple axes. The technique can be used until a performance criterion is met or for a fixed training period.

In certain embodiments, subjects are given a series of evoked pain stimuli in random order of intensity and asked to rate the intensity of the stimuli on a pain rating scale. The subject's responses are collected and analyzed for their consistency and reliability (e.g. for a stimuli of objective intensity X does the subject always report the subjective experience of Y, or a range from Y to Z?). Subjects additionally provide ratings of a naturalistic pain state or “index pain” (e.g. their pain from a chronic condition such as osteoarthritis or an acute pain such as from an injury) using the same rating scale and in terms of the evoked stimuli by means of cross-modality matching. Subjects are provided with feedback and undergo multiple cycles of evaluation and feedback to improve their ability to reliably report their pain states. This skill improves the quality of data the subject can provide in a clinical trial without biasing them towards positive or negative response to a treatment, therefore improving trial sensitivity and power.

### III. Baseline Evaluation

In certain embodiments, subjects are evaluated on their baseline ability to report evoked pain states accurately and use pain reporting scales consistently between evoked pain and clinical pain. In a preferred embodiment, this baseline evaluation is performed at the beginning of each training session.

Firstly, the subject's threshold and tolerance level for evoked pain stimuli is established. This can be done using any art-recognized methods. In a preferred embodiment, this is done by an ascending method of limits procedure in which the intensity of the stimulus is increased, either constantly or incrementally, until the subject reports that the stimulus has become painful. This is the threshold or lower bound. The stimulus is further increased until the subject reports that they cannot endure or tolerate any further increase. This is the tolerance or upper bound.

Secondly, the subject provides ratings of a natural index pain, such as their current pain from a chronic condition such as osteoarthritis or current pain from a recent surgical procedure or injury. Subjects rate this index pain on a standard scale (e.g. NRS) using Pain Matching. Pain Matching is accomplished by asking the subject to signal when a noxious stimulus (evoked pain) matches the intensity of their natural index pain. This can be done using any art-recognized methods. In a preferred embodiment, this is accomplished using a standard technique such as a staircase procedure, a method of limits, or method of adjustment. In the “staircase procedure” a stimulus is administered and the subject indicates if their index pain is more or less than the stimulus. The stimulus is then increased or decreased by an increment and assessed again. The increment is progressively narrowed until a minimum interval is reach. In the “method of limits” procedure there is a progressive increase of stimulus intensity from below threshold until the participant indicates a match (ascending method of limits) or a progressive decrease of stimulus intensity from above threshold until the participant indicates a match (descending method of limits). The “method of adjustment” procedure is similar to “method of limits”, however, the participant is allowed direct control of the stimulus intensity and can adjust it upward or downwards until it matches their natural index pain.

Thirdly, the subject undergoes a cycle of magnitude estimations of evoked pain stimuli between threshold and tolerance. Stimulus intervals are established, distributed between threshold and tolerance levels. The number of intervals may vary. In certain embodiments, the intervals are between 1 and 10, (e.g., between 5 and 9). Each level of stimulus is then administered multiple times. In certain embodiments, the varying each level of stimulus is administered between 1 and 10 times (e.g., between 3 and 7 times), in random order. The intervals and number of repetitions of each level may vary between programs based on the needs of the population. In certain embodiments, the intervals and number of repetitions of each level are fixed at or before the beginning of the training. For example, for a highly sensitive population such as subjects with fibromyalgia, a small number such as 5 intervals with only 3 repetitions for a total of 15 trials may be used, while a more robust population such as post-appendectomy patients may use 7 intervals and 7 repetitions for a total of 49 stimuli per cycle. Subjects provide a rating of the intensity of pain at each stimulus using a specified pain rating scale (e.g. NRS).

In certain embodiments, each evoked pain stimulus has a definable rate of increase and decrease (ramp) and a fixed peak duration. Subjects are instructed to rate the peak intensity of the stimulus. In certain embodiments, a minimum inter-stimulus interval between trials is fixed (this can dependent on stimulus modality, e.g., longer refractory periods may be required between thermal stimuli than electrical stimuli).

### IV. Subject Response Analyses

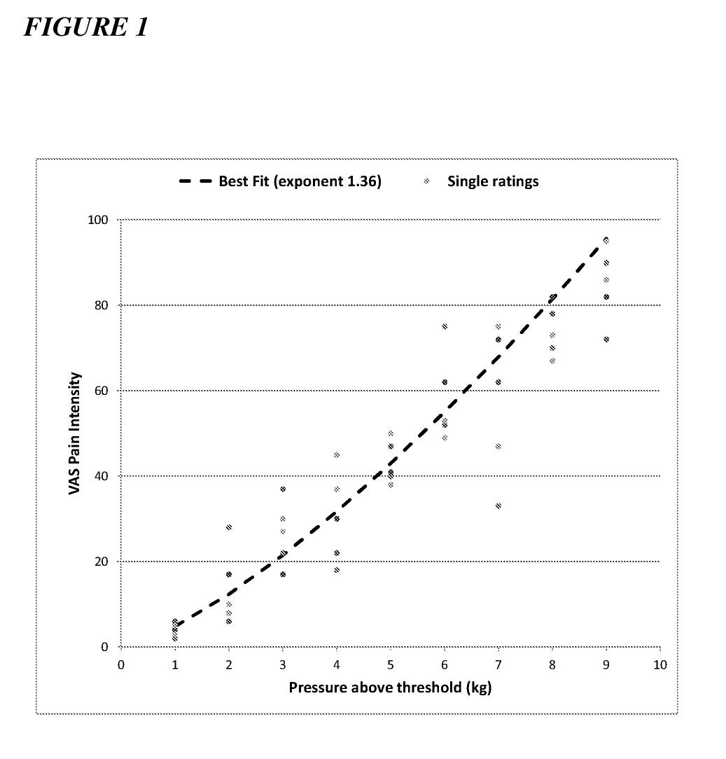

In general, a subject's threshold and tolerance for the evoked pain stimuli is analyzed as follows. Standard deviation of threshold, tolerance, and range are examined across training session to quantify stability over time using coefficients of variation (CoV), which is computed as standard deviation divided by mean. A subject's magnitude estimations are then used to compute a psychophysical profile (an exemplary psychophysical profile is depicted in FIG. 1). Data is centered and least-squares curve fitting is applied.

### Centering Data

Calculation of psychophysical function curves requires that ratings begin at threshold (or lower bound). Therefore, if a subject consistently rates the lowest stimulus at zero intensity the entire data set must be shifted (aka ‘left-censored’ or ‘centered’). This is accomplished by subtracting the highest stimulus intensity level at which pain of zero is reported from the objective stimulus quantification such that the first stimulus level is always 1. For example, a subject reporting thermal stimuli at intervals of 1 degree Celsius from 45 to 50 degrees reports zero pain at 45 and 46 degrees beginning to report pain only at 47 degrees C. The stimulus intensity for the data going into curve fitting becomes degrees C. minus 46. This is done to avoid a ‘tail’ to the data and shifting of the curve fit to accommodate sub-threshold stimuli.

Any device calibration or response scaling required by the device being used may be performed at this stage. For example, if the response scale is a 0-10 but the recording device reports 0-100 this conversion can be conducted simultaneously with data centering.

### Curve Fitting

Centered data are then fit to a least squares curve fit model. The least squares curve fitting is done using the following equation form:

$$Y = {Ax}^{B} where$$

$$b = \frac{n {Σ}_{i = 1}^{n} ( ln {x}_{i} ln {y}_{i} ) - {Σ}_{i - 1}^{n} ( ln {x}_{i} ) {Σ}_{i - 1}^{n}}{n {{Σ}_{i = 1}^{n} ( ln {x}_{i} )}^{2} - {( {Σ}_{i = 1}^{n} ln {x}_{i} )}^{2}}$$

$$a = \frac{{Σ}_{i = 1}^{n} ( ln {y}_{i} ) - b {Σ}_{i = 1}^{n} ( ln {x}_{i} )}{n}$$

$$B = b and A = {e}^{a}$$

### Triangulation

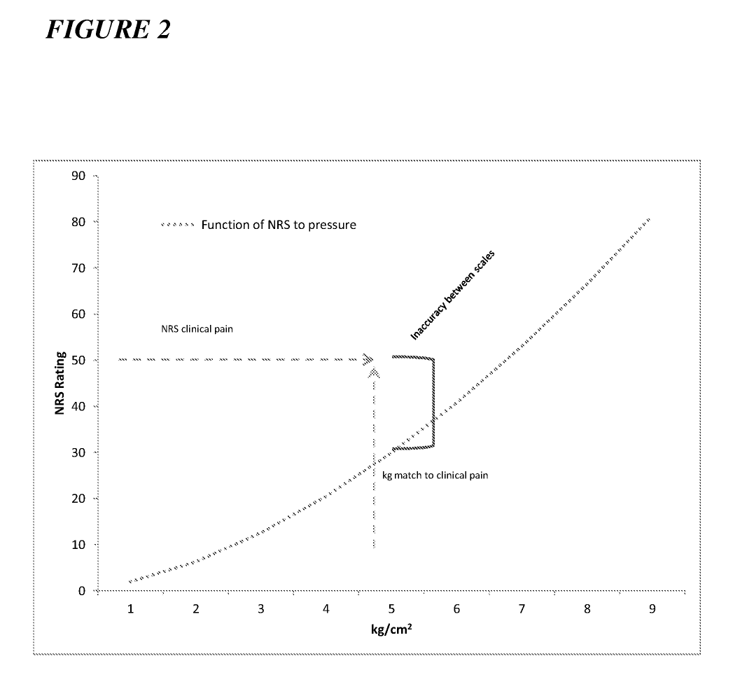

Comparison is made of how consistently a subject uses a response scale using a method called “triangulation”. By providing a standard scale rating of index pain, a stimulus matched rating of the same index pain and a standard scale rating of evoked stimuli, the subject has given three ratings that should theoretically converge. For example, using NRS ratings and pressure pain, a subject could report their index pain as 5 out of 10 (moderate pain) and match their index pain to a pressure intensity of 3 kg (saying 3 kg pressure causes pain equivalent to their index pain), but when rating the intensity of 3 kg of pressure on a 0-10 NRS they give an average rating of only 2. Such a result would indicate an inconsistency in scale use by the subject, because, according to the psychophysical profile established by the subject's rating of blinded stimuli, 3 kg of pressure was not as painful as their rated index pain. This is illustrated in FIG. 2 and is quantified by the residual between the point where index pain standard scale and Pain Match ratings intersect and a vertical line dropped to the psychophysical function. For example, if a subject rates theirindex pain at 6/10, Pain Matches the index pain to pressure at 4 kg, and the psychophysical function indicates that 4 kg of pressure are rated at 3.5, the residual (inconsistency between scale use) would be 2.5 (6−3.5).

### Quantification of Report Reliability

Report reliability within an assessment cycle is quantified by: 1) average Coefficient of Variation (CoV) where CoV is calculated for each non-zero stimulus level and averaged; 2) R² fit to the least squares model; 3) average Intraclass Correlation Coefficient (ICC) calculated from all non-zero stimulus levels; and 4) the triangulation residual.

### V. Training Feedback

In certain embodiments, after baseline evaluation, subjects receive training feedback based upon their performance. Feedback can be given using any method, including without limitation, written or oral methods. In a particular embodiment, data figures analogous to FIGS. 1 and 2, herein, are generated from the subject's actual reporting data and shown to them, along with idealized samples to illustrate accurate and inaccurate scale use. The data is reviewed with the subject by the trainer conducting the session and their attention is called to areas of high variability and/or inconsistency. For example, a subject is shown where a thermal stimulus (e.g., a 48° stimulus) was inaccurately rated as more painful than a cooler stimulus (e.g., a 46° stimulus). The subject is further instructed to pay attention to their pain state, keep in mind how they have used the scales previously, and try to be consistent. Such feedback is provided after each training cycle.

### VI. Training Cycles and Session Scheduling

The number of training cycles conducted in a single session and the total number of sessions conducted may vary between training programs. At least 2 cycles of evaluation with feedback must be completed (one for baseline and a second to establish any change), but more may be conducted as desired. In certain embodiments, training sessions are separated by a minimum of about 2 days and a maximum of about 14 days (e.g., about 3, 4, 5, 6, 7, 8, 9, 10, 11, 12 or 13 days).

In certain embodiments, training cycles are not separated by more than 1 hour within a session. Sessions can be repeated as necessary until a minimum performance criterion is met (e.g. until subject's triangulation residual is <2 and R² is >0.9) or for a pre-specified number of sessions (e.g. 4 weekly sessions on consecutive weeks prior to study enrollment) depending on desired use.

The number of training cycles within a session may also be varied according to the burden and demands of the target population. For example, a generally young and vigorous post-surgical subject may have a narrow window of opportunity but high tolerance for training (e.g., 2 sessions 3 days apart, each session containing 4 training cycles) whereas a highly sensitive elderly subject with chronic pain may have as many sessions as necessary to meet performance criterion (e.g., sessions scheduled weekly and only containing 1 training cycle per session).

### VI. Methods of Identifying an Accurate Pain Reporting Subject

In another aspect, the present invention provides methods of identifying an accurate pain reporting subject. Such methods generally comprise: determining the reported pain threshold and tolerance levels of the subject in response to an evoked pain stimulus; determining the reported pain of the subject in response to a natural index pain using a standard pain reporting scale; determining the response profile of the subject to an array of noxious stimuli using a standard pain reporting scale, wherein the noxious stimuli evoke pain that is between the pain threshold and tolerence levels of the subject; and determining the pain reporting accuracy and reliability of the subject by quantification and analysis of reported pain of the subject, wherein an accurate/reliable pain reporting subject would have pain reporting accuracy above a desired threshold accuracy.

Any art-recognized method of quantification and analysis of the reported pain of the subject can be employed. In certain embodiments, the accuracy of the subject's pain reporting accuracy is determined using the Coefficient of Variation (see e.g., Reed, J. F., Lynn, F., & Meade, B. D. (2002). Use of coefficient of variation in assessing variability i of quantitative assays. Clin Diagn Lab Immuno. 9(6), 1235-1239, which is incorporated herein by reference in its entirety). In a particular embodiment, a Coefficient of Variation of less than 1 (e.g., about 0.9. 0.8, 0.7, 0.6, 0.5, 0.4, 0.3, 0.2, or 0.1) identifies a subject as an accurate pain reporter.

In certain embodiments, the accuracy of the subject's pain reporting accuracy is determined using the Intraclass Correlation Coefficient (see e.g., Shrout, P. E., & Fleiss, J. L. (1979). Intraclass correlations: Uses in assessing rater reliability. Psychological Bulletin, 86, 420-428, which is incorporated herein by reference in its entirety). In a particular embodiment, an Intraclass Correlation Coefficient of greater than 0.95 (e.g., about 0.96. 0.97, 0.98, or 0.99) identifies a subject as an accurate pain reporter.

In certain embodiments, the accuracy of the subject's pain reporting accuracy is determined using an R² curve fit statistic from a least squares fit to psychophysical function (power law) (see e.g., Stevens, S. S. (1961) The psychophysics of sensory function. In Rosenblith, W. A. (ed.) Sensory Communications, 1-33, which is incorporated herein by reference in its entirety). In a particular embodiment, an R² of greater than 0.5 (e.g., about 0.6. 0.7, 0.8, 0.9, or 1.0) identifies a subject as an accurate pain reporter.

In certain embodiments, the accuracy of the subject's pain reporting accuracy is determined using the Residual between predicted and actual pain ratings using a ‘triangulation’ method (see e.g., Gracely, R, & Kwilosz, D. M. (1988). The Descriptor Differential Scale: Applying psychophysical principles to clinical pain assessment. Pain, 35, 279-288; and Doctor, J. N., Slater, M. A., & Atkinson, J. H. (1995). The descriptor differential scale of pain intensity: An evaluation of item and scale properties. Pain, 61, 251-260, both which is incorporated herein by reference in their entirety). In a particular embodiment, a triangulation residual of less than 15% (e.g., about 14, 13, 12, 11, 10, 9, 8, 7, 6, 5, 4, 3, 2, or 1%) of the response scale being used (e.g. less than 15 if a 0-100 mm VAS is used as the standard response scale) identifies a subject as an accurate pain reporter.

### VII. Pain Rating Scales

The methods disclosed herein can use any art-recognized pain rating scale or measure. Suitable scales include, without limitation, standard numerical rating scales (NRS) or visual analog scales (VAS), and any quantitative pain report method, including measures of specific aspects of pain (e.g. the McGill Pain Questionnaire item for intensity of burning pain specifically).

### VIII. Evoked Pain Modality

Any evoked pain modality can be used in the methods disclosed herein. In certain embodiments, evoked pain is applied to the subject using a device that can, via mechanical or electronic control, reliably exert a variable intensity stimulus of a noxious nature within a range that is both painful and safe. Examples of painful modalities include, but are not limited to, heat, cold, pressure, electrical stimulation, chemical (e.g. capsaicin), ischemic, or visceral pain. Suitable common devices include the Medoc TSA-II neuro sensory analyzer (Medoc, Israel), which can apply controlled heat stimuli via a thermode in contact with the skin or the Multimodal Automated Sensory Testing (MAST, UMich), which can apply calibrated pressure stimuli to the thumbnail. In a preferred embodiment, the device is capable of delivering repeated stimuli at fixed levels without variable intervention of a human agent (e.g. a hand-held dolorimeter with pressure exerted by a human operator would be unacceptable). In a preferred embodiment, the device is capable of exerting sufficient stimulus intensity to exceed pain thresholds for subjects but not so much as to cause potential injury. In a preferred embodiment, the device has acceptable safety functions in place such that a subject may terminate any stimulus at any time.

1. A method of training a subject to report pain comprising:
a) determining the reported pain threshold and tolerance levels of the subject in response to an evoked pain stimulus;
b) determining the reported pain of the subject in response to a natural index pain using a standard pain reporting scale;
c) determining the response profile of the subject to noxious stimuli using a standard pain reporting scale, wherein the noxious stimuli intensity are between the pain threshold and tolerance levels of the subject;
d) determining the pain reporting accuracy and/or reliability of the subject by analysis of the data collected in (a), (b), and (c);
e) providing instructional feedback to the subject regarding the accuracy and reliability of their pain reporting; and
f) repeating steps (a) to (e) one or more times.

2. The method of claim 1, wherein step (f) is repeated until a desired reporting accuracy is achieved.

3. The method of claim 1, wherein step (f) is repeated a predetermined number of times.

4. A method of identifying an accurate pain reporting subject:
a) determining the reported pain threshold and tolerance levels of the subject in response to an evoked pain stimulus;
b) determining the reported pain of the subject in response to a natural index pain using a standard pain reporting scale;
c) determining the response profile of the subject noxious stimuli using a standard pain reporting scale, wherein the noxious stimuli evoke pain that is between the pain threshold and tolerance levels of the subject;
d) determining the pain reporting accuracy and/or reliability of the subject by analysis of data obtained in (a), (b), and (c), wherein an accurate pain reporting subject is identified by having a pain reporting accuracy and/or reliability above a desired threshold.

5. The method of claim 1, wherein the pain threshold and tolerance levels of the subject are determined in response to a mechanical pressure or thermal stimulus.

6. The method of claim 1, wherein the index pain is knee pain from osteoarthritis.

7. The method claim 1, wherein the noxious stimuli include mechanical pressure.

8. The method claim 1, wherein the noxious stimuli are applied in a random order of intensity.

9. The method of claim 1, the noxious stimuli are applied in discreet interval levels, evenly spaced between the subject's threshold and tolerance levels.

10. The method of claim 9, wherein the noxious stimuli are applied in 5 to 9 interval levels.

11. The method of claim 9, wherein each interval level of noxious stimuli is applied between 3 and 7 times to the subject during a single session.

12. The method of claim 1, wherein the standard pain reporting scale is a numerical rating scale (NRS) or visual analog scale (VAS).

13. The method claim 1, wherein the pain reporting accuracy and/or reliability of the subject is determined using a the Coefficient of Variation, Intraclass Correlation Coefficient, R² curve fit statistic from a least squares fit to psychophysical function, and/or the Residual between the predicted and actual pain ratings using a ‘triangulation’ method.

14. The method of claim 4, wherein an accurate pain reporting subject is identified by having a Coefficient of Variation of less than 1, an Intraclass Correlation Coefficient of greater than 0.8, an R² of greater than 0.5, and/or a triangulation residual of less than 20% of the range of the response scale being used.
